(19) United States
(12) Patent Application Publication
(10) Pub. No.: US 2006/0189873 A1
(43) Pub. Date: Aug. 24, 2006

(54) Illustrations of sodium intakes & hypertension

(76) Inventors: Anuthep Benja-Athon, New York, NY (US); Nontawan Benja-Athon, Port Washington, NY (US)

Correspondence Address: ANUTHEP BENJA-ATHON, M.D.;Pain Management, Acupuncture Physical Therapy, 210 E 36th St., New York, NY, 10016, US

(21) Appl. No.: 11/064,349

(22) Filed: Feb. 24, 2005

(51) Int. Cl. A61B 5/02

(52) U.S. Cl. 600/500

(57) ABSTRACT

An instantaneous feedback for modulating an individual's systolic and diastolic blood pressures and the consumptions of sodium. The feedback is the outcomes of mathematical equations leading to a calculation or a plurality of calculations involving sodium consumptions. A plurality of values, a graph or a plurality of graphs are also the outcome of the above equations and calculations. The outcomes is the tallies of sodium consumptions, a graph or a plurality of graphs showing the sodium intakes, and sodium consumptions corresponding values of systolic and diastolic blood pressures.

### FIELD OF INVENTION

Feedback for modulating sodium consumptions and blood pressures.

### BACKGROUND OF THE INVENTION

The first objective of the present invention is to provide a means for modulating blood pressures by determining the sodium consumptions of an individual.

The second objective of the present invention is to provide a means for modulating blood pressures by an illustration means for showing a relationship of sodium consumptions and blood pressures.

The third objective of the present invention is to provide a means for modulating blood pressures by an illustration means for showing a relationship of extrapolated quantities of sodium consumptions and blood pressures.

The fourth objective of the present invention is to provide a means for modulating blood pressures by a means for guiding and regulating an individual's blood pressures and by a means for guiding and regulating said individual's sodium consumptions.

The fifth objective of the present invention is to provide a means for reducing or eliminating high blood pressures which are significant risk factors for heart diseases and attacks and strokes.

There exists a relationship between sodium intakes and systolic and diastolic blood pressures in individual—humans or animals. The increased consumptions of sodium caused increased systolic and diastolic blood pressures in a normotensive or hypertensive individual. Corollary, the reductions of sodium consumptions caused the falls of the systolic and diastolic blood pressures in a normotensive or hypertensive individual.

Americans and most people in the developed and developing nations consume too much salt or sodium chloride daily. Almost all processed foods have added sodium which is added to foods during the processes of manufacturing and/or processing. Almost all people do not know that they are consuming excess sodium each day. The lack of a means for an individual to determine the sodium consumptions, a means for an individual to relate said individual's sodium consumptions to said individual's blood pressures and a means for an individual to extrapolate quantities of sodium and said individual's extrapolated blood pressures is a fundamental impediment to effectively and safely affect, control and regulate said individual's blood pressures by regulating the sodium intakes.

So, more than 60 million Americans are hypertensive. While they continue to eat and load up on sodium every day, essentially, their treatments comprise the life-long consumptions of numerous types of anti-hypertensive drugs such as diuretics which cause the body to excrete sodium through the kidneys.

Said objectives of the present invention are achieved by the means described infra.

### SUMMARY OF THE INVENTION

Americans consume too much salt daily contributing to high blood pressure in more than 60 million Americans. Almost all, if not all, people are unaware of the excessive salt intakes and heretofore have no means for controlling or regulating the salt consumptions.

A means for modulating an individual's systolic and diastolic blood pressures by modulating the consumptions of sodium. Specifically, the present invention provides an instantaneous feedback means for affecting an individual's sodium consumptions adaptable to effect an individual's modulation of systolic and diastolic blood pressures. Therefore, an individual can monitor thence regulate or modulate his or her intakes of sodium—an equivalence of a drug—to physiologically constantly regulate or modulate his or her systolic and diastolic blood pressures.

Said means is an outcome of the use of mathematical equations to a obtain a plurality of values, a graph or a plurality of graphs relating to sodium or salt consumptions per unit time and correspoding systolic and diasytolic blood pressures of an individual. The outcomes are the tallies of sodium consumptions relating to values of said blood pressures. To enhance the power of the invention, a graph or a plurality of graphs showing the sodium intakes, and sodium consumptions corresponding values of systolic and diastolic blood pressures. Furthermore, in addition to the above, said mathematical means and outcomes are used to instantaneously show, calculate, predict or extrapolate an individual's extrapolated value or plurality of values—in numbers, graph or a plurality of graphs—of systolic and diastolic blood pressures relative to a quantity or quantities of sodium consumed or yet to be consumed.

### DESCRIPTION OF THE PREFERRED EMBODIMENTS

The present invention provides a unique and powerful means for modulating an individual's systolic and diastolic blood pressures relating to the modulation of the consumptions of sodium—commonly known as salt—adaptable to help or effect an individual's modulations of said pressures.

Specifically, the present invention provides an instantaneous feedback means for affecting an individual's sodium consumptions adaptable to effect an individual's modulation of systolic and diastolic blood pressures. Said means is constantly and accessible on-demand by an individual on 24×7 yielding an instantaneous response comprising a value, a plurality of values, an illustration, a plurality of illustrations, a graph and/or a plurality of graphs relating to sodium consumptions and blood pressures. In other words, the present invention allows an individual the instantaneous means for constantly monitoring thence regulating or modulating his or her intakes of salt or sodium—an equivalence of a drug—to constantly regulate or modulate his or her systolic and diastolic blood pressures. The benefits for an individual and the nation are immense.

Said instantaneous feedback means for affecting an individual's sodium consumptions comprises a mathematical means for tallying the amounts of sodium consumed, being or to be consumed by an individual. The tallies quantifying said consumptions are used to inform an individual. One of the preferred embodiments comprises a mathematical means expressed as a function of FOOD ITEMₙ, NUMBER OF PORTIONS OF FOOD ITEMₙ, QUANTITY OF SODIUM PER PORTION OF FOOD ITEMSₙ and TIME, where 1=n>1. For example, one simplified representation of said preferred embodiments is FOOD ITEMₙ,×NUMBER OF PORTIONS OF FOOD ITEMₙ×QUANTITY OF SODIUM PER PORTION OF FOOD ITEMSₙ per unit time TIME. Various mathematical expressions or formulas can be used to achieve the quantification of the sodium consumptions relating to blood pressures of an individual within the spirit of the invention and the scope of the claims.

One of the outcomes of said mathematical expressions, equations or calculations is the tally or tallies of sodium consumptions per unit time.

Another outcome of said mathematical expressions, equations or calculations is the illustrations of the sodium consumptions relative to time of an individual. The outcomes are expressed in a number or a plurality of numbers showing the milligrams or grams of sodium being consumed in a given unit of time such as hours, days, weeks or months. Better yet, in addition to the above, said outcomes are further shown in a graph or a plurality of graphs showing the sodium intakes relating to time units such as hours, days, weeks or months.

Yet another outcome of said mathematical expressions, equations or calculations is the illustrations of the sodium consumptions relative to time of an individual corresponding to individual's systolic and diastolic blood pressures. In other words, said outcome shows a number of sodium consumption corresponding to a value of systolic and diastolic blood pressures. However, in addition to the above, it is more certain that said outcome shows a plurality of values of sodium consumptions corresponding to a plurality of values of systolic and diastolic blood pressures. Better yet, in addition to the above, said outcome are further shown in a graph or a plurality of graphs showing the sodium consumptions relating to time units such as hours, days, weeks or months and corresponding values of systolic and diastolic blood pressures.

Furthermore, in addition to the above, said mathematical means and outcomes are used to instantaneously show, calculate, predict or extrapolate an individual's extrapolated value or plurality of values of systolic and diastolic blood pressures relative to a quantity or quantities of sodium consumed or yet to be consumed.

According to the above fundamentals, the present invention also provides the accounts—in numbers and illustrations in graphs or the likes—of sodium contents in individual food portion such as, but not limited to, a serving, spoon, slice, cup, individual sodium contents in individual portions of individual food brands such as, but not limited to brand XYZ or brand No-Name. Furthermore, if an individual can supply with said food portion of a brand product, the present invention provides a means for extrapolating the sodium content said brand portion.

The individual or individuals in this invention are construed as human or humans and animal or animals. It is further understood that salt is interchangeably used to connote sodium chloride or sodium which are the terms commonly use. Moreover, various weight systems such as, but not limited to, the metric system of milligrams or grams or other systems such as ounces or pounds can be used without departing from the spirit of the invention and the scope of the claims.

Although various preferred embodiments of this invention have been described, it will be appreciated by those skilled in the art that adaptations and variations may be made without departing from the spirit of the invention and the scope of the claims.

1. A means for modulating an individual's systolic and diastolic blood pressures relating to the consumptions of sodium commonly known as salt adaptable to help an individual's modulations of said pressures comprises:
a mathematical means for quantifying sodium consumptions;
an illustration means for showing an outcome of said mathematical means;
an illustration means for showing a tally on sodium consumptions;
an illustration means for showing quantity of sodium consumptions;
an illustration means for showing the tally on sodium consumptions;
II an illustration means for showing sodium balances;
an illustration means for showing individual sodium contents in individual food portions;
an illustration means for showing individual sodium contents in individual portions of individual food brands;
an extrapolating means for determining individual sodium contents in individual portions of individual food brands;
a means for relating individual food portions and individual sodium contents;
a means for relating individual food brands and individual sodium contents;
a means for relating an individual's sodium consumptions and said individual's systolic and diastolic blood pressures; and
an extrapolation means for illustrating the relationship of quantities of sodium and an individual's extrapolated systolic and diastolic blood pressures.

2. The means for modulating an individual's systolic and diastolic blood pressures according to claim 1 wherein sodium consumptions in said mathematical means for quantifying sodium consumptions is a function of FOOD ITEMₙ, NUMBER OF PORTIONS OF FOOD ITEMₙ, QUANTITY OF SODIUM PER PORTION OF FOOD ITEMSₙ and TIME, where 1=n>1.

3. The means for modulating an individual's systolic and diastolic blood pressures according to claim 1 wherein said illustration means for showing the tally of sodium consumptions comprises a graph means for illustrating the sodium intakes relating to time units.

4. The means for modulating an individual's systolic and diastolic blood pressures according to claim 1 wherein said illustration means for showing the relationship of an individual's sodium consumptions and said individual's systolic and diastolic blood pressures comprises a graph means for illustrating the relationship of an individual's systolic and diastolic blood pressures and said individual sodium consumptions.

5. The means for modulating an individual's systolic and diastolic blood pressures according to claim 1 wherein said extrapolation means for illustrating the relationship of quantities of sodium and an individual's systolic and diastolic blood pressures comprises a graph means for extrapolating individual systolic and diastolic blood pressures and individual quantities of sodium.

6. The means for modulating an individual's systolic and diastolic blood pressures according to claim 1 wherein said means for relating an individual's sodium consumptions and said individual's systolic and diastolic blood pressures comprises an illustration means for showing the relationship of an individual's sodium consumptions and said individual's systolic and diastolic blood pressures.

7. The illustration means according to claim 6 includes a graph means for showing a relationship of an individual's sodium consumptions and said individual's systolic and diastolic blood pressures.

8. The illustration means according to claim 6 includes an extrapolating means for showing a relationship of an individual's sodium consumptions and predictable individual's systolic and diastolic blood pressures.

9. A means for regulating an individual's sodium consumptions relating to an individual's modulations of systolic and diastolic blood pressures comprises:
an illustration means for showing an outcome of said mathematical means;
an illustration means for showing a tally on sodium consumptions;
an illustration means for showing quantity of sodium consumptions;
an illustration means for showing the tally on sodium consumptions;
an illustration means for showing sodium balances;
an illustration means for showing individual sodium contents in individual food portions;
an illustration means for showing individual sodium contents in individual portions of individual food brands;
an extrapolating means for determining individual sodium contents in individual portions of individual food brands;
a means for relating individual food portions and individual sodium contents;
a means for relating individual food brands and individual sodium contents;
a means for relating an individual's sodium consumptions and said individual's systolic and diastolic blood pressures; and
an extrapolation means for illustrating the relationship of quantities of sodium and an individual's extrapolated systolic and diastolic blood pressures.

10. An instantaneous feedback means for affecting an individual's sodium consumptions adaptable to effect an individual's modulation of systolic and diastolic blood pressures comprises:
an illustration means for showing an outcome of said mathematical means;
an illustration means for showing a tally on sodium consumptions;
an illustration means for showing quantity of sodium consumptions;
an illustration means for showing the tally on sodium consumptions;
an illustration means for showing sodium balances;
an illustration means for showing individual sodium contents in individual food portions;
an illustration means for showing individual sodium contents in individual portions of individual food brands;
an extrapolating means for determining individual sodium contents in individual portions of individual food brands;
a means for relating individual food portions and individual sodium contents;
a means for relating individual food brands and individual sodium contents;
a means for relating an individual's sodium consumptions and said individual's systolic and diastolic blood pressures; and
an extrapolation means for illustrating the relationship of quantities of sodium and an individual's extrapolated systolic and diastolic blood pressures.

11. The instantaneous feedback means for affecting an individual's sodium consumptions according to claim 10 wherein sodium consumptions in said mathematical means for quantifying sodium consumptions is a function of FOOD ITEMₙ, NUMBER OF PORTIONS OF FOOD ITEMₙ, QUANTITY OF SODIUM PER PORTION OF FOOD ITEMSₙ and TIME, where 1=n>1.

12. The instantaneous feedback means for affecting an individual's sodium consumptions according to claim 10 wherein said an illustration means for showing the tally of sodium consumptions comprises a graph means for illustrating the sodium intakes relating to time units.

13. The instantaneous feedback means for affecting an individual's sodium consumptions according to claim 10 wherein said illustration means for showing the relationship of an individual's sodium consumptions and said individual's systolic and diastolic blood pressures comprises a graph means for illustrating the relationship of an individual's systolic and diastolic blood pressures and said individual sodium consumptions.

14. The instantaneous feedback means for affecting an individual's sodium consumptions according to claim 10 wherein said extrapolation means for illustrating the relationship of quantities of sodium and an individual's extrapolated systolic and diastolic blood pressures comprises a graph means for extrapolating individual systolic and diastolic blood pressures and individual quantities of sodium.
